(19) United States
(12) Patent Application Publication
(10) Pub. No.: US 2023/0203545 A1
(43) Pub. Date: Jun. 29, 2023

(54) METHOD FOR CONTROLLED PRODUCTION OF GLYCERIC ACID

(71) Applicants: Inner Mongolia Kingdomway Pharceutical Limited, Inner Mongolia (CN); Xiamen Kingdomway Group Company, Fujian (CN); Kingdomway Biotech. (Jiangsu) Co., Ltd., Jiangsu (CN)

(72) Inventors: Zhichun Zhu, Fujian (CN); Guanghuang Zhan, Fujian (CN); Changhua Dai, Fujian (CN); Luming Xu, Fujian (CN); Huijuan Zheng, Fujian (CN); Biqin Chen, Fujian (CN); Dan Li, Fujian (CN)

(21) Appl. No.: 17/974,870

(22) Filed: Oct. 27, 2022

(30) Foreign Application Priority Data
Dec. 24, 2021 (CN) 202111597381.2

(51) Int. Cl. C12P 7/42; C12N 1/20

(52) U.S. Cl. CPC C12P 7/42; C12N 1/20; C12N 2500/60; C12N 2500/74; C12N 2500/14

(57) ABSTRACT

A method for controlled production of glyceric acid includes, during the glyceric acid fermentation process, real-time online monitoring of at least one of the respiratory quotient and redox potential in the fermentation liquid as well as the fermentation process specific growth rate, and controlling their values within the following ranges: 0.1˜1.5 for respiratory quotient, −300˜50 mV for redox potential, 0.05˜0.8 for specific growth rate. Process parameters for this method are controlled within predetermined ranges by online monitoring at least one of respiratory quotient (RQ) and redox potential as well as the specific growth rate in the fermentation process, thereby meeting the growth requirements of bacteria, effectively promoting the glycerol metabolism and synthesis and improving the conversion rate.

### CROSS-REFERENCE TO RELATED APPLICATIONS

This application claims priority pursuant to 35 U.S.C. 119(a) to Chinese Patent Application No. 202111597381.2 filed Dec. 24, 2021, which application is incorporated herein by reference in its entirety.

### TECHNICAL FIELD

The invention belongs to the field of glyceric acid production, and in particular relates to a method for controlled production of glyceric acid.

### BACKGROUND

Glyceric acid is a natural organic compound found in a variety of plants such as peanut, tomato, apple, banana and grape, and is a phytochemical component. Glyceric acid exists in the human body as a metabolite, and glyceric acid phosphate derivatives are important intermediates in the process of glycolysis. According to literature reports, glyceric acid in the human body can make gastric cells stimulated by ethanol to promote ethanol catabolism. There are also literature reports that glyceric acid has functions of cholesterol activity and liver stimulant, and can also be used as a transport carrier for drugs. Therefore, glyceric acid has great application value.

At present, the methods for producing glyceric acid mainly include chemical synthesis method and microbial fermentation method. Among them, the chemical synthesis method mainly uses a strong oxidant to oxidize glycerol to generate glyceric acid. The chemical synthesis method is cumbersome and produces many glycerol derivatives. The conversion rate is not high and the product is complex, which is not conducive to the separation and extraction of the product, and also forms a lot of wastes that have negative impact on the environment, and thus it has high cost and high energy consumption. The microbial fermentation method has mild production conditions, high substrate utilization, simple and easy-to-control process, high product purity, convenient separation and extraction in the later stage, little environmental pollution, low cost, and is conducive to industrial production. There are not many reports on the production of glyceric acid by microbial fermentation, in which only laboratory scale is disclosed, the reported glyceric acid fermentation level is low, fermentation period is long, conversion rate is low, and thus it cannot meet the requirements of industrial production.

The currently reported glyceric acid fermentation process mainly controls oxygen supply and feeding through dissolved oxygen feedback. This controlled process has the problem of lag, and its product yield and conversion rate are unstable. At present, there is no report on online monitoring of respiratory quotient (RQ) and redox potential and as well as fermentation process specific growth rate for the feedback regulation of glyceric acid fermentation process.

### SUMMARY

The purpose of the present invention is to overcome the problems of long fermentation period, low product yield and low conversion rate in existing glyceric acid fermentation methods, and provide a method for controlled production of glyceric acid with short fermentation period, stable high yield and high conversion rate.

Specifically, the method for controlled production of glyceric acid provided by the present invention comprises the following step: during the glyceric acid fermentation process, performing real-time and online monitoring of at least one of a respiratory quotient and a redox potential in a fermentation broth as well as a fermentation process specific growth rate, and controlling the values thereof within the following ranges: 0.1˜1.5 for respiratory quotient, −300˜50 mV for redox potential, 0.05˜0.8 for specific growth rate.

In a preferred embodiment, the respiratory quotient is controlled in four stages, and the control method is as follows: during the fermentation period from hour 0 to hour 24, the respiratory quotient in the fermentation broth is controlled at 0.1˜1.1; during the fermentation period from hour 24 to hour 48, the respiratory quotient in the fermentation broth is controlled at 0.2˜1.3; during the fermentation period from hour 48 to hour 72, the respiratory quotient in the fermentation broth is controlled at 0.1˜0.8; during the fermentation period from hour 72 to the end of fermentation, the respiratory quotient in the fermentation broth is controlled at 0.1˜0.4.

In a preferred embodiment, the respiratory quotient is controlled in eight stages, and the control method is as follows: during the fermentation period from hour 0 to hour 12, the respiratory quotient in the fermentation broth is controlled at 0.1-0.8; during the fermentation period from hour 12 to hour 24, the respiratory quotient in the fermentation broth is controlled at 0.1-1.1; during the fermentation period from hour 24 to hour 36, the respiratory quotient in the fermentation broth is controlled at 0.2˜1.3; during the fermentation period from hour 36 to hour 48, the respiratory quotient in the fermentation broth is controlled at 0.3˜1.0; during the fermentation period from hour 48 to hour 60, the respiratory quotient in the fermentation broth is controlled at 0.3˜0.8; during the fermentation period from hour 60 to hour 72, the respiratory quotient in the fermentation broth is controlled at 0.1˜0.6; during the fermentation period from hour 72 to hour 84, the respiratory quotient in the fermentation broth is controlled at 0.1˜0.4; during the period from hour 84 to the end of fermentation, the respiratory quotient in the fermentation broth is controlled at 0.1˜0.2.

In a preferred embodiment, the redox potential is controlled in four stages, and the control method is as follows: during the fermentation period from hour 0 to hour 24, the redox potential in the fermentation broth is controlled at −100˜50 mV; during the period from hour 24 to hour 48, the redox potential in the fermentation broth is controlled at −300˜−50 mV; during the fermentation period from hour 48 to hour 72, the redox potential in the fermentation broth is controlled at −250˜−100 mV; during the fermentation period from hour 72 to the end of fermentation, the redox potential in the fermentation broth is controlled at −200˜−50 mV.

In a preferred embodiment, the redox potential is controlled in eight stages, and the control method is as follows: during the fermentation period from hour 0 to hour 12, the redox potential in the fermentation broth is controlled at −50˜50 mV; during the period from hour 12 to hour 24, the redox potential in the fermentation broth is controlled at −100˜−50 mV; during the fermentation period from hour 24 to hour 36, the redox potential in the fermentation broth is controlled at −200˜−50 mV; during the fermentation period from hour 36 to hour 48, the redox potential in the fermentation broth is controlled at −300˜−100 mV; during the fermentation period from hour 48 to hour 60, the redox potential in the fermentation broth is controlled at −200˜−100 mV; during the fermentation period from hour 60 to hour 72, the redox potential in the fermentation broth is controlled at −250˜−100 mV; during the fermentation period from hour 72 to hour 84, the redox potential in the fermentation broth is controlled at −200˜−50 mV; during the fermentation period from hour 84 to the end of fermentation, the redox potential in the fermentation broth is controlled at −150˜−50 mV.

In a preferred embodiment, the specific growth rate is controlled in four stages, and the control method is as follows: during the fermentation period from hour 0 to hour 24, the specific growth rate is controlled at 0.05˜0.8; during the fermentation period from hour 24 to hour 48, the specific growth rate is controlled at 0.2˜0.6; during the fermentation period from hour 48 to hour 72, the specific growth rate is controlled at 0.2˜0.5; during the fermentation period from hour 72 to the end of fermentation, the specific growth rate is controlled at 0.05˜0.3.

In a preferred embodiment, the specific growth rate is controlled in eight stages, and the control method is as follows: during the fermentation period from hour 0 to hour 12, the specific growth rate is controlled at 0.05˜0.6; during the fermentation period from hour 12 to hour 24, the specific growth rate is controlled at 0.2˜0.8; during the fermentation period from hour 24 to hour 36, the specific growth rate is controlled at 0.2˜0.6; during the fermentation period from hour 36 to hour 48, the specific growth rate is controlled at 0.3˜0.5; during the fermentation period from hour 48 hour to hour 60, the specific growth rate is controlled at 0.2˜0.5; during the fermentation period from hour 60 to hour 72, the specific growth rate is controlled at 0.15˜0.4; during the fermentation period from hour 72 to hour 84, the specific growth rate is controlled at 0.1˜0.3; during the period from hour 84 to the end of fermentation, the specific growth rate is controlled at 0.05˜0.15.

In a preferred embodiment, the numerical ranges of the respiratory quotient and redox potential in the fermentation broth as well as the fermentation process specific growth rate are controlled by adjusting at least one of rotation speed, air flow rate and tank pressure. When the respiratory quotient and redox potential in the fermentation broth as well as the fermentation process specific growth rate cannot be maintained within the predetermined ranges by adjusting the rotation speed, air flow rate and tank pressure, a feed solution is supplemented, and the feed solution is at least one selected from the group consisting of calcium oxide, calcium hydroxide and calcium carbonate, and the feed solution is used in an amount allowing the fermentation broth to have a final concentration of calcium ions of 0.5 to 15 g/L.

In a preferred embodiment, the strain used for the glyceric acid fermentation is at least one selected from Acetobacter tropicalis, Gluconobacter thailandicus and Gluconobacter frateurii.

In a preferred embodiment, the production process of glyceric acid comprises the following steps:

S1. Seed activation: A plate medium is prepared, its pH value is adjusted to 5.5 to 7.5, and then it is sterilized at 121˜123° C. for 20˜30 minutes; the components of the plate medium are as follows: sodium chloride 1˜10 g/L, peptone 1˜15 g/L, yeast extract powder 1˜15 g/L and agar powder 10˜20 g/L; the bacterial liquid is pipetted from the seed preservation tube and subjected to gradient dilution, the diluted bacterial suspension is pipetted and transferred to the sterilized plate medium, and cultured at 28˜38° C. for 2˜5 days to obtain matured single colonies;

S2. Shake flask culture: A shake flask medium is prepared, its pH value is adjusted to 5.5 to 7.5, and then it is sterilized at 121˜123° C. for 20˜30 minutes; the components of the shake flask medium are as follows: sodium chloride 1˜10 g/L, peptone 1˜15 g/L and yeast extract powder 1˜15 g/L; 1˜20 single colonies are picked from the matured plate medium and inoculated to the sterilized shake flask medium, the shake flask culture conditions comprise: culture temperature of 28˜38° C., rotation speed of 150˜250 rpm, and culture cycle of 4˜48 hours; when the wet weight of the bacteria reaches 1˜20 g/L, it is transferred to a seed tank, and the inoculation amount is controlled at 0.1˜10%;

S3. Seed tank culture: A seed medium is prepared, its pH value is adjusted to 5.5 to 7.5, and then it is sterilized at 121˜123° C. for 20 to 30 minutes; the components of the seed medium are as follows: glucose 1˜15 g/L, sodium chloride 1˜10 g/L, peptone 1˜15 g/L and yeast extract powder 1˜15 g/L; the seed tank culture conditions comprise: culture temperature of 28˜38° C., tank pressure of 0.025˜0.08 MPa, aeration ratio of 0.2˜2 VVM, rotation speed of 100˜500 rpm, culture cycle of 4˜48 hours, when the wet weight of the bacteria reaches 1˜20 g/L, it is transferred to a fermentation tank, and the inoculation amount is controlled at 5˜30%;

S4. Fermentation culture: A fermentation medium is prepared, its pH value is adjusted to 5.5˜7.5, and then it is sterilized at 121˜123° C. for 20˜30 minutes; the components of the fermentation medium are as follows: potassium dihydrogen phosphate 1˜10 g/L, sodium chloride 1˜10 g/L, yeast extract powder 1˜15 g/L, zinc chloride 0.005˜0.5 g/L, manganese sulfate 0.0001˜0.5 g/L and glycerol 20˜300 g/L; the fermentation culture conditions comprise: culture temperature of 28˜38° C., tank pressure of 0.02˜0.08 MPa, aeration ratio of 0.2˜2 VVM, and rotation speed of 50˜1000 rpm; during the fermentation culture process, an alkali is added to control the pH value of the fermentation system at 3.5˜7.5, a fed-batch medium is supplemented to control the carbon source concentration at 0.1˜100 g/L and the nitrogen source concentration at 0.01˜2 g/L in the fermentation system.

In a preferred embodiment, in step S4, the alkali is sodium hydroxide and/or ammonia water.

In a preferred embodiment, in step S4, the carbon source is at least one selected from glucose, sucrose and glycerol.

In a preferred embodiment, in step S4, the nitrogen source is at least one selected from ammonium sulfate, yeast extract and peptone.

In a preferred embodiment, in step S4, the components of the fed-batch medium are as follows: glycerol concentration 200˜600 g/L, ammonia water 200˜280 g/L, sodium hydroxide 100˜400 g/L, ammonium sulfate 1˜50 g/L and yeast extract powder 1˜50 g/L.

In a preferred embodiment, in step S4, when the growth rate of the product is significantly slowed down or the staining of bacterial cells is light, which is used as a standard for terminating the fermentation, HPLC is used to measure the concentration of glyceric acid in the fermentation broth.

The key of the present invention is that the process parameters are feedback controlled by online monitoring at least one of the respiratory quotient and redox potential as well as the fermentation process specific growth rate, so as to control the process parameters within predetermined ranges, to meet the growth requirements of the bacteria, to effectively promote the metabolic synthesis of glyceric acid, and to improve the conversion rate. The method for controlled production of glyceric acid provided by the invention is suitable for different equipment scales and strains, effectively ensures the consistency of cell growth and product metabolism in the complex oleic acid production process, shortens the fermentation period, achieves a fermentation level of stable high yield and high conversion rate, good process reproducibility, and achieves the purpose of effectively reducing production cost.

### DETAILED DESCRIPTION

In a preferred embodiment of the present invention, the respiratory quotient and redox potential in fermentation broth as well as the fermentation process specific growth rate are controlled in 4 stages (i.e., hour 0 to hour 24, hour 24 to hour 48, hour 48 to hour 72, and hour 72 to the end of fermentation). It should be noted that hour 0 to hour 24 comprises: 1^{st} hour, 2^{nd} hour, 3^{rd} hour, 4^{th} hour . . . 21^{st} hour, 22^{nd} hour, 23^{rd} hour, 24^{th} hour, a total of 24 hours; hour 24 to hour 48 comprises 25^{th} hour, 26^{th} hour, 27^{th} hour, 28^{th} hour . . . 45^{th} hour, 46^{th} hour, 47^{th} hour, 48^{th} hour, a total of 24 hours; hour 48 to hour 72 comprises 49^{th} hour, 50^{th} hour, 51^{st} hour, 52^{nd} hour . . . 69^{th} hour, 70^{th} hour, 71^{th} hour, 72^{nd} hour, a total of 24 hours; and hour 72 to the end of fermentation comprises: 73^{rd} hour, 74^{th} hour . . . the end of fermentation.

In a preferred embodiment of the present invention, the respiratory quotient and redox potential in fermentation broth as well as the fermentation process specific growth rate are controlled in 8 stages (i.e., hour 0 to hour 12, hour 12 to hour 24, hour 24 to hour 36, hour 36 to hour 48, hour 48 to hour 60, hour 60 to hour 72, hour 72 to hour 84, and hour 84 to the end of fermentation). It should be noted that hour 0 to hour 12 comprises: 1^{st} hour, 2^{nd} hour, 3^{rd} hour, 4^{th} hour . . . 9^{th} hour, 10^{th} hour, 11^{th} hour, 12^{th} hour, a total of 12 hours; hour 12 to hour 24 comprises: 13^{th} hour, 14^{th} hour, 15^{th} hour, 16^{th} hour . . . 21^{st} hour, 22^{nd} hour, 23^{rd} hour, 24^{th} hour, a total of 12 hours; hour 24 to hour 36 comprises 25^{th} hour, 26^{th} hour, 27^{th} hour, 28^{th} hour . . . 33^{rd} hour, 34^{th} hour, 35^{th} hour, 36^{th} hour, a total of 12 hours; hour 36 to hour 48 comprises 37^{th} hour, 38^{th} hour, 39^{th} hour, 40^{th} hour . . . 45^{th} hour, 46^{th} hour, 47^{th} hour, 48^{th} hour, a total of 12 hours; hour 48 to hour 60 comprises 49^{th} hour, 50^{th} hour, 51^{st} hour, 52^{nd} hour . . . 57^{th} hour, 58^{th} hour, 59^{th} hour, 60^{th} hour, a total of 12 hours; hour 60 to hour 72 comprises 61^{st} hour, 62^{nd} hour, 63^{rd} hour, 64^{th} hour . . . 69^{th} hour, 70^{th} hour, 71^{st} hour, 72^{nd} hour, a total of 12 hours; hour 72 to hour 84 comprises 73^{rd} hour, 74^{th} hour, 75^{th} hour, 76^{th} hour . . . 81^{st} hour, 82^{nd} hour, 83^{rd} hour, 84^{th} hour, a total of 12 hours; and hour 84 to the end of fermentation comprises: 85^{th} hour, 86^{th} hour . . . the end of fermentation.

The respiratory quotient (RQ) is a response of the intracellular metabolic flow of microorganisms on macroscopic parameters, which can reflect the changes in the utilization of substrates and the synthesis of products and by-products during the fermentation process. In the fermentation process of glyceric acid, the respiratory quotient in the fermentation broth is controlled at 0.1˜1.5 (e.g., 0.1, 0.2, 0.3, 0.4, 0.5, 0.6, 0.7, 0.8, 0.9, 1.0, 1.1, 1.2, 1.3, 1.4, 1.5, etc.), which can promote the conversion of substrates and synthesis of products, minimize the accumulation of by-products, and improve the level of fermentation.

In the present invention, the respiratory quotient RQ is calculated by the following formula:

$$R Q = \frac{CER}{OUR}$$

$$CER = \frac{F i n}{V ⋆ 22.4} * ( {C}_{{CO}_{2 i n}} - \frac{* {C}_{{CO}_{2 i n}}}{1 - ( {C}_{{CO}_{2 out}} + {C}_{{O}_{2 out}}} )$$

$$OUR = \frac{{F}_{i n}}{V ⋆ 22.4} * ( {C}_{{o}_{2 i n}} - \frac{* {C}_{{o}_{2 i n}}}{1 - ( {C}_{{o}_{2 out}} + {C}_{{co}_{2 out}} )} )$$

Fᵢₙ: Air inflow (L/h or m³/h);

V: volume (L or m³) of fermentation broth;

C_{O2in}: oxygen concentration (%) of intake air;

Cco₂ᵢₙ: carbon dioxide concentration (%) of intake air;

[symbol] : inert gas concentration (%) of intake air;

Co₂ₒᵤₜ: oxygen concentration (%) of exhaust gas of fermentation broth;

Cco₂ₒᵤₜ: carbon dioxide concentration (%) of exhaust gas of fermentation broth;

wherein, for Co₂ᵢₙ, [symbol] , Co₂ₒᵤₜ, Cco₂ₒᵤₜ, their concentrations are monitored in real-time by a mass spectrometer.

In a preferred embodiment of the present invention, the respiratory quotient in fermentation broth is controlled in 4 stages, and the control method is as follows: during the fermentation period from hour 0 to hour 24, the respiratory quotient in fermentation broth is controlled at 0.1˜1.1, such as 0.1, 0.2, 0.3, 0.4, 0.5, 0.6, 0.7, 0.8, 0.9, 1.0, 1.1, etc.; during the fermentation period from hour 24 to hour 48, the respiratory quotient in fermentation broth is controlled at 0.2˜1.3, such as 0.2, 0.3, 0.4, 0.5, 0.6, 0.7, 0.8, 0.9, 1.0, 1.1, 1.2, 1.3, etc.; during the fermentation period from hour 48 to hour 72, the respiratory quotient in fermentation broth is controlled at 0.1˜0.8, such as 0.1, 0.2, 0.3, 0.4, 0.5, 0.6, 0.7, 0.8, etc.; during the period from hour 72 to the end of fermentation, the respiratory quotient in fermentation broth is controlled at 0.1˜0.4, such as 0.1, 0.2, 0.3, 0.4, etc.

In a preferred embodiment of the present invention, the respiratory quotient in fermentation broth is controlled in 8 stages, and the control method is as follows: during the fermentation period from hour 0 to hour 12, the respiratory quotient in fermentation broth is controlled at 0.1˜0.8, such as 0.1, 0.2, 0.3, 0.4, 0.5, 0.6, 0.7, 0.8, etc.; during the fermentation period from hour 12 to hour 24, the respiratory quotient in fermentation broth is controlled at 0.1˜1.1, such as 0.1, 0.2, 0.3, 0.4, 0.5, 0.6, 0.7, 0.8, 0.9, 1.0, 1.1, etc.; during the fermentation period from hour 24 to hour 36, the respiratory quotient in fermentation broth is controlled at 0.2˜1.3, such as 0.2, 0.3, 0.4, 0.5, 0.6, 0.7, 0.8, 0.9, 1.0, 1.1, 1.2, 1.3, etc.; during the fermentation period from hour 36 to hour 48, the respiratory quotient in fermentation broth is controlled at 0.3˜1.0, such as 0.3, 0.4, 0.5, 0.6, 0.7, 0.8, 0.9, 1.0, etc.; during the fermentation period from hour 48 to hour 60, the respiratory quotient in fermentation broth is controlled at 0.3˜0.8, such as 0.3, 0.4, 0.5, 0.6, 0.7, 0.8, etc.; during the fermentation period from hour 60 to hour 72, the respiratory quotient in fermentation broth is controlled at 0.1˜0.6, such as 0.1, 0.2, 0.3, 0.4, 0.5, 0.6, etc.; during the fermentation period from hour 72 to hour 84, the respiratory quotient in fermentation broth is controlled at 0.1˜0.4, such as 0.1, 0.2, 0.3, 0.4, etc.; during the fermentation period from hour 84 to the end of fermentation, the respiratory quotient in fermentation broth is controlled at 0.1˜0.2, such as 0.1, 0.2, etc.

In the fermentation process of glyceric acid, the redox potential in fermentation broth must be controlled at −300˜50 mV, such as −300 mV, −250 mV, −200 mV, −150 mV, −100 mV, −50 mV, 0 mV, 10 mV, 20 mV, 30 mV, 40 mV, 50 mV, etc. When the redox potential in fermentation broth is lower than −300 mV, it will affect the production of bacteria, thereby affecting the synthesis of glyceric acid; when the redox potential in fermentation broth is higher than 50 mV, the bacteria will overgrow, by-products will increase, substrate interest rate will be low, and raw material costs will increase. In the present invention, the redox potential is monitored online in real time by a redox potential electrode.

In a preferred embodiment of the present invention, the redox potential is controlled in 4 stages, and the control method is as follows: during the fermentation period from hour 0 to hour 24, the redox potential in fermentation broth is controlled at −100˜50 mV, such as −100 mV, −90 mV, −80 mV, −70 mV, −60 mV, −50 mV, −40 mV, −30 mV, −20 mV, −10 mV, 0 mV, 10 mV, 20 mV, 30 mV, 40 mV, 50 mV, etc.; during the fermentation period from hour 24 to hour 48, the redox potential in fermentation broth is controlled at −300˜−50 mV, such as −300 mV, −280 mV, −250 mV, −220 mV, −200 mV, −180 mV, −150 mV, −120 mV, −100 mV, −80 mV, −50 mV, etc.; during the fermentation period from hour 48 to hour 72, the redox potential in fermentation broth is controlled at −250˜−100 mV, such as −250 mV, −220 mV, −200 mV, −180 mV, −150 mV, −120 mV, −100 mV, etc.; during the fermentation period from 72 h to the end of fermentation, the redox potential in fermentation broth is controlled at −200˜−50 mV, such as −200 mV, −180 mV, −150 mV, −120 mV, −100 mV, −80 mV, −50 mV, etc.

In a preferred embodiment of the present invention, the redox potential is controlled in 8 stages, and the control method is as follows: during the fermentation period from hour 0 to hour 12, the redox potential in fermentation broth is controlled at −50˜50 mV, such as −50 mV, −40 mV, −30 mV, −20 mV, −10 mV, 0 mV, 10 mV, 20 mV, 30 mV, 40 mV, 50 mV, etc.; during the fermentation period from hour 12 to hour 24, the redox potential in fermentation broth is controlled at −100˜−50 mV, such as −100 mV, −90 mV, −80 mV, −70 mV, −60 mV, −50 mV, etc.; during the fermentation period from hour 24 to hour 36, the redox potential in fermentation broth is controlled at −200˜−50 mV, such as −200 mV, −190 mV, −180 mV, −170 mV, −160 mV, −150 mV, −140 mV, −130 mV, −120 mV, −110 mV, −100 mV, −90 mV, −80 mV, −70 mV, −60 mV, −50 mV, etc.; during the fermentation period from hour 36 to hour 48, the redox potential in fermentation broth is controlled at −300˜−100 mV, such as −300 mV, −290 mV, −280 mV, −270 mV, −260 mV, −250 mV, −240 mV, −230 mV, −220 mV, −210 mV, −200 mV, −190 mV, −180 mV, −170 mV, −160 mV, −150 mV, −140 mV, −130 mV, −120 mV, −110 mV, −100 mV, etc.; during the fermentation period from hour 48 to hour 60, the redox potential in fermentation broth is controlled at −200˜−100 mV, such as −200 mV, −190 mV, −180 mV, −170 mV, −160 mV, −150 mV, −140 mV, −130 mV, −120 mV, −110 mV, −100 mV, etc.; during the fermentation period from hour 60 to hour 72, the redox potential in fermentation broth is controlled at −250˜−100 mV, such as −250 mV, −240 mV, −230 mV, −220 mV, −210 mV, −200 mV, −190 mV, −180 mV, −170 mV, −160 mV, −150 mV, −140 mV, −130 mV, −120 mV, −110 mV, −100 mV, etc.; during the fermentation period from hour 72 to hour 84, the redox potential in fermentation broth is controlled at −200˜−50 mV, such as −200 mV, −190 mV, −180 mV, −170 mV, −160 mV, −150 mV, −140 mV, −130 mV, −120 mV, −110 mV, −100 mV, −90 mV, −80 mV, −70 mV, −60 mV, −50 mV, etc.; during the fermentation period from hour 84 to the end of fermentation, the redox potential in fermentation broth is controlled at −150˜−50 mV, such as −150 mV, −140 mV, −130 mV, −120 mV, −110 mV, −100 mV, −90 mV, −80 mV, −70 mV, −60 mV, −50 mV, etc.

The specific growth rate refers to an increased cell mass per unit mass of cells per unit time, and the unit is s⁻¹. The specific growth rate can reflect the bacterial cell growth rate and metabolic intensity from the side, and the specific growth rate affects the product conversion rate. In the fermentation process of glyceric acid, the specific growth rate is controlled at 0.05˜0.8 (e.g., 0.05, 0.1, 0.2, 0.3, 0.4, 0.5, 0.6, 0.7, 0.8, etc.), so as to maximize the synthesis of glyceric acid from the substrate, improve the fermentation level and conversion rate. When the specific growth rate is greater than 0.8, the bacterial cell growth is too fast, the substrate consumption is too large, and the product conversion rate is low; when the specific growth rate is less than 0.05, the bacterial cell growth is too slow, which will affect the glyceric acid synthesis rate.

In the present invention, specific

$$growth rate = \frac{Ln ( N {t}_{2} ) - Ln ( N {t}_{1} )}{{t}_{2} - {t}_{1}}$$

t₁: fermentation start time point, h;
t₂: fermentation cycle, h;
Nt₂: dry weight of bacteria when fermentation reaches time point t₂, g;
Nt₁: dry weight of bacteria at the start of fermentation time point t₁, g;
wherein, Nt₁ and Nt₂ are monitored in real time by a live cell electrode and the specific growth rate is calculated.

In a preferred embodiment of the present invention, the specific growth rate is controlled in 4 stages, and the control method is as follows: during the fermentation period from hour 0 to hour 24 h, the specific growth rate is controlled at 0.05˜0.8, such as 0.05, 0.1, 0.2, 0.3, 0.4, 0.5, 0.6, 0.7, 0.8, etc.; during the fermentation period from hour 24 to hour 48, the specific growth rate is controlled at 0.2˜0.6, such as 0.2, 0.3, 0.4, 0.5, 0.6, etc.; during the fermentation period from hour 48 to hour 72, the specific growth rate is controlled at 0.2˜0.5, such as 0.2, 0.3, 0.4, 0.5, etc.; during the fermentation period from hour 72 to the end of fermentation, the specific growth rate is controlled at 0.05˜0.3, such as 0.05, 0.1, 0.2, 0.3, etc.

In a preferred embodiment of the present invention, the specific growth rate is controlled in 8 stages, and the control method is as follows: during the fermentation period from hour 0 to hour 12, the specific growth rate is controlled at 0.05˜0.6, such as 0.05, 0.1, 0.2, 0.3, 0.4, 0.5, 0.6, etc.; during the fermentation period from hour 12 to hour 24, the specific growth rate is controlled at 0.2˜0.8, such as 0.2, 0.3, 0.4, 0.5, 0.6, 0.7, 0.8, etc.; during the fermentation period from hour 24 to hour 36, the specific growth rate is controlled at 0.2˜0.6, such as 0.2, 0.3, 0.4, 0.5, 0.6, etc.; during the fermentation period from hour 36 to hour 48, the specific growth rate is controlled at 0.3˜0.5, such as 0.3, 0.4, 0.5, etc.; during the fermentation period from hour 48 to hour 60, the specific growth rate is controlled at 0.2˜0.5, such as 0.2, 0.3, 0.4, 0.5, etc.; during the fermentation period from hour 60 to hour 72, the specific growth rate is controlled at 0.15˜0.4, such as 0.15, 0.2, 0.3, 0.4, etc.; during the fermentation period from hour 72 to hour 84, the specific growth rate is controlled at 0.1˜0.3, such as 0.1, 0.2, 0.3, etc.; during the fermentation period from hour 84 to the end of fermentation, the specific growth rate is controlled at 0.05˜0.15, such as 0.05, 0.1, 0.15, etc.

In the present invention, the strain used in the fermentation culture can be various existing strains suitable for producing glyceric acid, specific examples of which include but are not limited to at least one of: Acetobacter tropicalis (e.g., CGMCC1.3694), Gluconobacter thailandicus (e.g., NBRC100600), Gluconobacter frateurii (e.g., NBR3262) and the like.

During the fermentation process, at least one of the respiratory quotient and redox potential in the fermentation broth as well as the fermentation process specific growth rate is monitored in real time and used as feedback to the glyceric acid fermentation process, and the respiratory quotient, redox potential and specific growth rate are maintained by increasing or decreasing at least one of rotation speed, air flow and tank pressure. Furthermore, when the respiratory quotient, redox potential and specific growth rate cannot be maintained within the predetermined ranges by adjusting the rotation speed, air flow and tank pressure, a feed solution should be supplemented. The feed solution is at least one selected from the group consisting of calcium oxide, calcium hydroxide and calcium carbonate. In addition, the feed solution is preferably used in an amount such that the final concentration of calcium ions in the fermentation broth is 0.5˜15 g/L, such as 0.5, 1, 2, 3, 4, 5, 6, 7, 8, 9, 10, 11, 12, 13, 14, 15 g/L.

In addition, when glyceric acid reaches a certain concentration, it will inhibit the growth and metabolism of bacterial cells, and the synthesis rate of glyceric acid is slowed down, which affects the synthesis of glyceric acid product. At this time, the inhibition can also be eliminated by supplementing a feed solution, and the feed solution can be at least on selected from the group consisting of calcium chloride, calcium hydroxide, calcium carbonate, etc., thereby increasing the yield of glyceric acid.

The production process of glyceric acid provided by the present invention comprises seed activation, shake flask culture, seed tank culture and fermentation culture. There is no special restriction on the medium formula for each culture stage, and it can be a suitable conventional medium.

In a preferred embodiment, the glyceric acid fermentation comprises the following steps:

S1. Seed activation: A plate medium is prepared, its pH value is adjusted to 5.5 to 7.5, and then it is sterilized at 121˜123° C. for 20˜30 minutes; the components of the plate medium are as follows: sodium chloride 1˜10 g/L, peptone 1˜15 g/L, yeast extract powder 1˜15 g/L and agar powder 10˜20 g/L; the bacterial liquid is pipetted from the seed preservation tube and subjected to gradient dilution, the diluted bacterial suspension is pipetted and transferred to the sterilized plate medium, and cultured at 28˜38° C. for 2˜5 days to obtain matured single colonies;

S2. Shake flask culture: A shake flask medium is prepared, its pH value is adjusted to 5.5 to 7.5, and then it is sterilized at 121˜123° C. for 20˜30 minutes; the components of the shake flask medium are as follows: sodium chloride 1˜10 g/L, peptone 1˜15 g/L and yeast extract powder 1˜15 g/L; 1˜20 single colonies are picked from the matured plate medium and inoculated to the sterilized shake flask medium, the shake flask culture conditions comprise: culture temperature of 28˜38° C., rotation speed of 150˜250 rpm, and culture cycle of 4˜48 hours; when the wet weight of the bacteria reaches 1˜20 g/L, it is transferred to a seed tank, and the inoculation amount is controlled at 0.1˜10%;

S3. Seed tank culture: A seed medium is prepared, its pH value is adjusted to 5.5 to 7.5, and then it is sterilized at 121˜123° C. for 20 to 30 minutes; the components of the seed medium are as follows: glucose 1˜15 g/L, sodium chloride 1˜10 g/L, peptone 1˜15 g/L and yeast extract powder 1˜15 g/L; the seed tank culture conditions comprise: culture temperature of 28˜38° C., tank pressure of 0.025˜0.08 MPa, aeration ratio of 0.2˜2 VVM, rotation speed of 100˜500 rpm, culture cycle of 4˜48 hours, when the wet weight of the bacteria reaches 1˜20 g/L, it is transferred to a fermentation tank, and the inoculation amount is controlled at 5˜30%;

S4. Fermentation culture: A fermentation medium is prepared, its pH value is adjusted to 5.5˜7.5, and then it is sterilized at 121˜123° C. for 20˜30 minutes; the components of the fermentation medium are as follows: potassium dihydrogen phosphate 1˜10 g/L, sodium chloride 1˜10 g/L, yeast extract powder 1˜15 g/L, zinc chloride 0.005˜0.5 g/L, manganese sulfate 0.0001˜0.5 g/L and glycerol 20˜300 g/L; the fermentation culture conditions comprise: culture temperature of 28˜38° C., tank pressure of 0.02˜0.08 MPa, aeration ratio of 0.2˜2 VVM, and rotation speed of 50˜1000 rpm; during the fermentation culture process, an alkali is added to control the pH value of the fermentation system at 3.5˜7.5, a fed-batch medium is supplemented to control the carbon source concentration at 0.1˜100 g/L and the nitrogen source concentration at 0.01˜2 g/L in the fermentation system. Wherein, the alkali can be sodium hydroxide and/or ammonia water. Specific examples of the carbon source include, but are not limited to, at least one of glucose, sucrose, and glycerol. Specific examples of the nitrogen source include, but are not limited to, at least one of ammonium sulfate, yeast extract and peptone. In a preferred embodiment, the components of the fed-batch medium are as follows: glycerol concentration 200˜600 g/L, ammonia water 200˜280 g/L, sodium hydroxide 100˜400 g/L, ammonium sulfate 1˜50 g/L and yeast extract powder 1˜50 g/L. Generally, when the product growth rate is significantly slowed down or the staining of bacterial cells is light, which is used as the termination standard, the concentration of glyceric acid in the fermentation broth is measured by HPLC. Specifically, the concentration of glyceric acid can be measured by using a Shodex SH1011 chromatographic column made of stainless steel, in which the diameter of 8 mm, the length is 250 mm, the particle size is 6 m, the refractive index detector Agilent 1200 Series is used, the mobile phase is 5 mM H₂SO₄, the flow rate is 0.8 mL/min, the injection volume is 20 μL, and the column temperature is kept at 50° C. to make the glyceric acid retention time is maintained at about 11 minutes. In addition, the volume of the fermentation tank may be 0.5 L to 500 m³.

The present invention will be described in detail below by means of examples.

The strains used in Examples 1 to 15 were all Acetobacter tropicalis (CGMCC 1.3694).

### Example 1: Production Process for 100 L Tank Fermentation

S1. Seed activation: A little bacterial liquid was pipetted from the seed preservation tube for gradient dilution, a small amount of the diluted bacterial suspension was pipetted and transferred onto a plate medium, and cultured at 32° C. for 24 hours to obtain matured single colonies. Wherein, the components of the plate medium were as follows: sodium chloride 5 g/L, peptone 5 g/L, yeast extract powder 5 g/L, agar powder 20 g/L, the pH was adjusted to 6 and then it was sterilized at 121° C. for 25 min.

S2. Shake flask culture: 3 Single colonies were picked from the matured plate medium and transferred in a shake flask containing 100 mL of shake flask medium, in which the shake flask is a 1 L conical flask, it was placed on a shaker for culturing, the culture temperature was 32° C., the rotation speed was 220 rpm, the culture was carried out for 20 h, and when the wet weight of the cells reached 4 g/L, the cells were transferred to a seed tank for cultivation. Wherein, the components of the shake flask medium were as follows: sodium chloride 5 g/L, peptone 5 g/L, yeast extract powder 5 g/L, the pH was adjusted to 6 and then it was sterilized at 121° C. for 25 minutes.

S3. Seed tank culture: A seed medium was prepared, its pH was adjusted to 6, then it was sterilized at 121° C. for 25 minutes, and the shake flask seed liquid is inoculated to a 15 L seed tank at an inoculation amount of 1%, and the liquid filling volume was 9 L. The seed culture conditions comprised: culture temperature of 32° C., tank pressure of 0.05 MPa, aeration ratio of 1.5 VVM, rotation speed of 500 rpm, and the pH was controlled at about 7.5 by adding ammonia water during the culture process. The culture period was 24 h. When the wet weight reached 5 g/L, it was transferred to a fermentation tank for culture. Wherein, the components of the seed medium were as follows: glucose 10 g/L, sodium chloride 5 g/L, peptone 5 g/L, and yeast extract 5 g/L.

S4. Fermentation culture: A fermentation medium was prepared, adjusted to have a pH of 7.5, and sterilized at 121° C. for 25 minutes, and the seed liquid of the seed tank was inoculated to a 100 L fermentation tank at an inoculation amount of 10%, and the liquid filling volume was 50 L. The initial culture conditions comprised: culture temperature of 32° C., rotation speed of 200 rpm, aeration ratio of 0.5 VVM, tank pressure of 0.03 MPa, the pH was controlled at about 7.5 by supplementing ammonia water during the culture process, and 500 g/L glucose solution and 2 g/L ammonium sulfate were added in fed-batch manner during the culture process. Wherein, the components of the fermentation medium were as follows: potassium dihydrogen phosphate 2 g/L, sodium chloride 2 g/L, yeast extract powder 10 g/L, zinc chloride 0.015 g/L, manganese sulfate 0.05 g/L and glycerol 100 g/L.

During the fermentation culture process, the RQ concentration in the fermentation broth was monitored in real time to perform the feedback control of the process in real time, the RQ concentration was controlled at the following level by increasing or decreasing at least one of rotation speed, air flow and tank pressure, and when the respiratory quotient could not be maintained within the predetermined range through the rotation speed, air flow and tank pressure, calcium chloride was added to the system, and the final concentration of calcium ions in the fermentation broth was controlled at 2 g/L:

1) during the fermentation period from hour 0 to hour 12, the RQ in the fermentation broth was controlled at 0.1 to 1.1;

2) during the fermentation period from hour 24 to hour 48, the RQ in the fermentation broth was controlled at 0.2˜1.3;

3) during the fermentation period from hour 48 to hour 72, the RQ in the fermentation broth was controlled at 0.1˜0.8;

4) during the fermentation period from hour 72 to the end of fermentation, the RQ in the fermentation broth was controlled at 0.1˜0.4.

The fermentation was stopped when the growth rate of the product was significantly slowed down or the bacterial cells were lightly stained, and the glyceric acid content in the fermentation broth was measured by HPLC. When the fermentation was stopped, the glyceric acid content reached 124 g/L, and the conversion rate reached 47%.

### Example 2: Production Process for 5 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and sterilized at 121° C. for 25 minutes, and the seed liquid of the seed tank was transferred to a 5 m³ fermentation tank at an inoculation amount of 15%, and the liquid filling volume was 2.5 m³. The initial culture conditions comprised: culture temperature of 32° C., rotation speed of 150 rpm, aeration ratio of 0.5 VVM, tank pressure of 0.03 MPa, the pH was controlled at about 7.5 by supplementing ammonia water during the culture process, and 500 g/L glucose and 10 g/L yeast extract powder were added in fed-batch manner during the culture process. Wherein, the components of the fermentation medium were as follows: potassium dihydrogen phosphate 5 g/L, sodium chloride 5 g/L, yeast extract powder 10 g/L, zinc chloride 0.25 g/L, manganese sulfate 0.25 g/L and glycerol 150 g/L.

During the fermentation culture process, the RQ concentration in the fermentation broth was monitored in real time to perform the feedback control of the process in real time, the RQ concentration was controlled at the following level by increasing or decreasing at least one of rotation speed, air flow and tank pressure, and when the respiratory quotient could not be maintained within the predetermined range through the rotation speed, air flow and tank pressure, calcium hydroxide was added to the system, and the final concentration of calcium ions in the fermentation broth was controlled at 5 g/L:

1) during the fermentation period from 0 to hour 12, the RQ in the fermentation broth was controlled at 0.1˜0.8;

2) during the fermentation period from 12 to hour 24, the RQ in the fermentation broth was controlled at 0.1˜1.1;

3) during the fermentation period from 24 to hour 36, the RQ in the fermentation broth was controlled at 0.2˜1.3;

4) during the fermentation period from 36 to hour 48, the RQ in the fermentation broth was controlled at 0.3˜1.0;

5) during the fermentation period from 48 to hour 60, the RQ in the fermentation broth was controlled at 0.3˜0.8;

6) during the fermentation period from 60 to hour 72, the RQ in the fermentation broth was controlled at 0.1˜0.6;

7) during the fermentation period from 72 to hour 84, the RQ in the fermentation broth was controlled at 0.1˜0.4;

8) during the fermentation period from hour 84 to the end of fermentation, the RQ concentration in the fermentation broth was controlled at 0.1˜0.2.

The fermentation was stopped when the growth rate of the product was significantly slowed down or the bacterial cells were lightly stained, and the glyceric acid content in the fermentation broth was determined by HPLC. When the fermentation was stopped, the glyceric acid content reached 145 g/L, and the conversion rate reached 55%.

### Example 3: Production Process for 5 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and sterilized at 121° C. for 25 minutes, and the seed liquid of the seed tank was transferred to a 5 m³ fermentation tank at an inoculation amount of 15%, and the liquid filling volume was 2.5 m³. The initial culture conditions comprised: culture temperature of 32° C., rotation speed of 150 rpm, aeration ratio of 0.5 VVM, tank pressure of 0.03 MPa, the pH was controlled at about 7.5 by supplementing ammonia water during the culture process, and 500 g/L glucose and 10 g/L yeast extract powder were added in fed-batch manner during the culture process. Wherein, the components of the fermentation medium were as follows: potassium dihydrogen phosphate 5 g/L, sodium chloride 5 g/L, yeast extract powder 10 g/L, zinc chloride 0.25 g/L, manganese sulfate 0.25 g/L and glycerol 150 g/L.

During the fermentation culture process, the RQ concentration in the fermentation broth was monitored in real time to perform the feedback control of the process in real time, the RQ concentration was controlled at the following level by increasing or decreasing at least one of rotation speed, air flow and tank pressure, and when the respiratory quotient could not be maintained within the predetermined range through the rotation speed, air flow and tank pressure, calcium carbonate was added to the system, and the final concentration of calcium ions in the fermentation broth was controlled at 5 g/L:

1) during the fermentation period from 0 to hour 12, the RQ in the fermentation broth was controlled at 0.1˜0.8;

2) during the fermentation period from 12 to hour 24, the RQ in the fermentation broth was controlled at 0.1˜1.1;

3) during the fermentation period from 24 to hour 36, the RQ in the fermentation broth was controlled at 0.2˜1.6;

4) during the fermentation period from 36 to hour 48, the RQ in the fermentation broth was controlled at 0.3˜1.0;

5) during the fermentation period from 48 to hour 60, the RQ in the fermentation broth was controlled at 0.3˜0.9;

6) during the fermentation period from 60 to hour 72, the RQ in the fermentation broth was controlled at 0.2˜0.6;

7) during the fermentation period from 72 to hour 84, the RQ in the fermentation broth was controlled at 0.1˜0.4;

during the fermentation period from hour 84 to the end of fermentation, the RQ concentration in the fermentation broth was controlled at 0.1˜0.2.

The fermentation was stopped when the growth rate of the product was significantly slowed down or the bacterial cells were lightly stained, and the glyceric acid content in the fermentation broth was determined by HPLC. When the fermentation was stopped, the glyceric acid content reached 132 g/L, and the conversion rate reached 50%.

Compared with Example 2, the RQ concentration was not controlled within the required range during some stages, and the fermentation level and conversion rate of glyceric acid were lower than those of Example 2.

### Example 4: Production Process for 120 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 120 m³ fermentation tank at an inoculation amount of 20%, and the liquid filling volume was 60 m³. The initial culture conditions comprised: culture temperature of 30° C., rotation speed of 60 rpm, aeration ratio of 1.2 VVM, tank pressure of 0.03 MPa, the pH was controlled at about 5 by supplementing ammonia water during the culture process, and 450 g/L glucose and 3 g/L yeast extract powder were added in fed-batch manner during the culture process. Wherein, the components of the fermentation medium were as follows: potassium dihydrogen phosphate 5 g/L, sodium chloride 5 g/L, yeast extract powder 10 g/L, zinc chloride 0.25 g/L, manganese sulfate 0.25 g/L and glycerol 150 g/L.

During the fermentation culture process, the redox potential in the fermentation broth was monitored in real time to perform the feedback control of the process in real time, the redox potential was controlled at the following level by increasing or decreasing at least one of rotation speed, air flow and tank pressure, and when the redox potential could not be controlled within the predetermined range through the rotation speed, air flow and tank pressure, calcium hydroxide was added to the system, and the final concentration of calcium ions in the fermentation broth was controlled at 4 g/L:

1) during the fermentation period from hour 0 to hour 24, the redox potential in the fermentation broth was controlled at −100˜50 mV;

2) during the fermentation period from hour 24 to hour 48, the redox potential in the fermentation broth was controlled at −300˜−50 mV;

3) during the fermentation period from hour 48 to hour 72, the redox potential in the fermentation broth was controlled at −250˜−100 mV;

4) during the fermentation period from hour 72 to the end of fermentation, the redox potential in the fermentation broth was controlled at −200˜−50 mV.

When the growth rate of the product was significantly slowed down or the bacterial cells were lightly stained, the fermentation was stopped, and the glyceric acid content in the fermentation broth was determined by HPLC. When the fermentation was stopped, the glyceric acid content reached 126 g/L, and the conversion rate reached 48%.

### Example 5: Production Process for 120 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 120 m³ fermentation tank at an inoculation amount of 20%, and the liquid filling volume was 60 m³. The initial culture conditions comprised: culture temperature of 30° C., rotation speed of 60 rpm, aeration ratio of 1.5 VVM, tank pressure of 0.03 MPa, the pH was controlled at about 5 by supplementing ammonia water during the culture process, and 450 g/L glucose and 3 g/L yeast extract powder were added in fed-batch manner during the culture process. Wherein, the components of the fermentation medium were as follows: potassium dihydrogen phosphate 5 g/L, sodium chloride 5 g/L, yeast extract powder 10 g/L, zinc chloride 0.25 g/L, manganese sulfate 0.25 g/L and glycerol 150 g/L.

During the fermentation culture process, the redox potential in the fermentation broth was monitored in real time to perform the feedback control of the process in real time, the redox potential was controlled at the following level by increasing or decreasing at least one of rotation speed, air flow and tank pressure, and when the redox potential could not be controlled within the predetermined range through the rotation speed, air flow and tank pressure, calcium hydroxide was added to the system, and the final concentration of calcium ions in the fermentation broth was controlled at 4 g/L:

1) during the fermentation period from hour 0 to hour 12, the redox potential in the fermentation broth was controlled at −50˜50 mV;

2) during the fermentation period from hour 12 to hour 24, the redox potential in the fermentation broth was controlled at −100˜−50 mV;

3) during the fermentation period from hour 24 to hour 36, the redox potential in the fermentation broth was controlled at −200˜−50 mV;

4) during the fermentation period from hour 36 to hour 48, the redox potential in the fermentation broth was controlled at −300˜−100 mV;

5) during the fermentation period from hour 48 to hour 60, the redox potential in the fermentation broth was controlled at −200˜−100 mV;

6) during the fermentation period from hour 60 to hour 72, the redox potential in the fermentation broth was controlled at −250˜−100 mV;

7) during the fermentation period from hour 72 to hour 84, the redox potential in the fermentation broth was controlled at −200˜−50 mV;

8) during the fermentation period from hour 84 to the end of fermentation, the redox potential in the fermentation broth was controlled at −150˜−50 mV.

When the growth rate of the product was significantly slowed down or the bacterial cells were lightly stained, the fermentation was stopped, and the glyceric acid content in the fermentation broth was determined by HPLC, and when the fermentation was stopped, the glyceric acid content reached 148 g/L, and the conversion rate reached 55%.

### Example 6: Production Process for 120 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 120 m³ fermentation tank at an inoculation amount of 20%, and the liquid filling volume was 60 m³. The initial culture conditions comprised: culture temperature of 30° C., rotation speed of 60 rpm, aeration ratio of 1.5 VVM, tank pressure of 0.03 MPa, the pH was controlled at about 5 by supplementing ammonia water during the culture process, and 450 g/L glucose and 3 g/L yeast extract powder were added in fed-batch manner during the culture process. Wherein, the components of the fermentation medium were as follows: potassium dihydrogen phosphate 5 g/L, sodium chloride 5 g/L, yeast extract powder 10 g/L, zinc chloride 0.25 g/L, manganese sulfate 0.25 g/L and glycerol 150 g/L.

During the fermentation culture process, the redox potential in the fermentation broth was monitored in real time to perform the feedback control of the process in real time, the redox potential was controlled at the following level by increasing or decreasing at least one of rotation speed, air flow and tank pressure, and when the redox potential could not be controlled within the predetermined range through the rotation speed, air flow and tank pressure, calcium hydroxide was added to the system, and the final concentration of calcium ions in the fermentation broth was controlled at 4 g/L:

1) during the fermentation period from hour 0 to hour 12, the redox potential in the fermentation broth was controlled at −50˜50 mV;

2) during the fermentation period from hour 12 to hour 24, the redox potential in the fermentation broth was controlled at −150˜−50 mV;

3) during the fermentation period from hour 24 to hour 36, the redox potential in the fermentation broth was controlled at −200˜−50 mV;

4) during the fermentation period from hour 36 to hour 48, the redox potential in the fermentation broth was controlled at −350˜−100 mV;

5) during the fermentation period from hour 48 to hour 60, the redox potential in the fermentation broth was controlled at −200˜−100 mV;

6) during the fermentation period from hour 60 to hour 72, the redox potential in the fermentation broth was controlled at −250˜−100 mV;

7) during the fermentation period from hour 72 to hour 84, the redox potential in the fermentation broth was controlled at −200˜−50 mV;

8) during the fermentation period from hour 84 to the end of fermentation, the redox potential in the fermentation broth was controlled at −150˜−50 mV.

When the growth rate of the product was significantly slowed down or the bacterial cells were lightly stained, the fermentation was stopped, and the glyceric acid content in the fermentation broth was determined by HPLC, and when the fermentation was stopped, the glyceric acid content reached 134 g/L, and the conversion rate reached 51%.

Compared with Example 5, the redox potential was not controlled within the range required by the process during some stages, and the fermentation level and conversion rate of glyceric acid were lower than those of Example 5.

### Example 7: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 12%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 1250 g/L.

During the fermentation culture process, the specific growth rate was monitored in real time to perform the feedback control of the process in real time, the specific growth rate was controlled at the following level by increasing or decreasing at least one of rotation speed, air flow and tank pressure, and when the specific growth rate could not be controlled within the predetermined range through the rotation speed, air flow and tank pressure, calcium hydroxide was added to the system, and the final concentration of calcium ions in the fermentation broth was controlled at 6 g/L:

1) during the fermentation period from hour 0 to hour 24, the specific growth rate was controlled at 0.05˜0.8;

2) during the fermentation period from hour 24 to hour 48, the specific growth rate was controlled at 0.2˜0.6;

3) during the fermentation period from hour 48 to hour 72, the specific growth rate was controlled at 0.2˜0.5;

4) during the fermentation period from hour 72 to the end of fermentation, the specific growth rate was controlled at 0.05˜0.3.

When the growth rate of the product was significantly slowed down or the bacterial cells were lightly stained, the fermentation was stopped, and the glyceric acid content in the fermentation broth was determined by HPLC; when the fermentation was stopped, the glyceric acid content reached 125 g/L, and the conversion rate reached 48%.

### Example 8: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 12%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 120 g/L.

During the fermentation culture process, the specific growth rate in the fermentation broth was monitored in real time to perform the feedback control of the process in real time, the specific growth rate was controlled at the following level by increasing or decreasing at least one of rotation speed, air flow and tank pressure, and when the specific growth rate could not be controlled within the predetermined range through the rotation speed, air flow and tank pressure, calcium hydroxide was added to the system, and the final concentration of calcium ions in the fermentation broth was controlled at 4 g/L:

1) during the fermentation period from hour 0 to hour 12, the specific growth rate was controlled at 0.05˜0.6;

2) during the fermentation period from hour 12 to hour 24, the specific growth rate was controlled at 0.2˜0.8;

3) during the fermentation period from hour 24 to hour 36, the specific growth rate was controlled at 0.2˜0.6;

4) during the fermentation period from hour 36 to hour 48, the specific growth rate was controlled at 0.3˜0.5;

5) during the fermentation period from hour 48 to hour 60, the specific growth rate was controlled at 0.2˜0.5;

6) during the fermentation period from hour 60 to hour 72, the specific growth rate was controlled at 0.15˜0.4;

7) during the fermentation period from hour 72 to hour 84, the specific growth rate was controlled at 0.1˜0.3;

8) during the fermentation period from hour 84 to the end of fermentation, the specific growth rate was controlled at 0.05˜0.15.

The fermentation was stopped when the growth rate of the product was significantly slowed down or the bacterial cells were lightly stained, and the glyceric acid content in the fermentation broth was determined by HPLC; when the fermentation was stopped, the glyceric acid content reached 149 g/L and the conversion rate reached 56%.

### Example 9: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 12%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 120 g/L.

During the fermentation culture process, the specific growth rate in the fermentation broth was monitored in real time to perform the feedback control of the process in real time, the specific growth rate was controlled at the following level by increasing or decreasing at least one of rotation speed, air flow and tank pressure, and when the specific growth rate could not be controlled within the predetermined range through the rotation speed, air flow and tank pressure, calcium hydroxide was added to the system, and the final concentration of calcium ions in the fermentation broth was controlled at 6 g/L:

1) during the fermentation period from hour 0 to hour 12, the specific growth rate was controlled at 0.05˜0.6;

2) during the fermentation period from hour 12 to hour 24, the specific growth rate was controlled at 0.2˜1.0;

3) during the fermentation period from hour 24 to hour 36, the specific growth rate was controlled at 0.2˜0.8;

4) during the fermentation period from hour 36 to hour 48, the specific growth rate was controlled at 0.3˜0.5;

5) during the fermentation period from hour 48 to hour 60, the specific growth rate was controlled at 0.15˜0.5;

6) during the fermentation period from hour 60 to hour 72, the specific growth rate was controlled at 0.15˜0.4;

7) during the fermentation period from hour 72 to hour 84, the specific growth rate was controlled at 0.1˜0.3;

8) during the fermentation period from hour 84 to the end of fermentation, the specific growth rate was controlled at 0.05˜0.15.

The fermentation was stopped when the growth rate of the product was significantly slowed down or the bacterial cells were lightly stained, and the glyceric acid content in the fermentation broth was determined by HPLC; when the fermentation was stopped, the glyceric acid content reached 135 g/L and the conversion rate reached 52%.

Compared with Example 8, the specific growth rate was not controlled within the range required by the process during some stages, and the fermentation level and conversion rate of glyceric acid were lower than those of Example 8.

### Example 10: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 20%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 120 g/L.

During the fermentation culture process, the RQ and redox potential in the fermented broth were monitored in real time to perform the feedback control of the process in real time, the RQ and redox potential were controlled at the following levels by increasing or decreasing at least one of rotation speed, air flow and tank pressure, and when the RQ and redox potential could not be maintained within the predetermined ranges through the rotation speed, air flow and tank pressure, calcium hydroxide was added to the system, and the final concentration of calcium ions in the fermentation broth was controlled at 9 g/L:

1) during the fermentation period from hour 0 to hour 24, the RQ in the fermentation broth was controlled at 0.1˜1.1, and the redox potential in the fermentation broth was controlled at −100˜50 mV;

2) during the fermentation period from hour 24 to hour 48, the RQ in the fermentation broth was controlled at 0.2˜1.3, and the redox potential in the fermentation broth was controlled at −300˜−50 mV;

3) during the fermentation period from hour 48 to hour 72, the RQ in the fermentation broth was controlled at 0.1˜0.8, and the redox potential in the fermentation broth was controlled at −250˜−100 mV;

4) during the fermentation period from hour 72 to the end of fermentation, the RQ in the fermentation broth was controlled at 0.1˜0.4, and the redox potential in the fermentation broth was controlled at −200˜−50 mV.

The fermentation was stopped when the growth rate of the product was significantly slowed down or the bacterial cells were lightly stained, and the glyceric acid content in the fermentation broth was determined by HPLC. When the fermentation was stopped, the glyceric acid content reached 151 g/L, and the conversion rate reached 57%.

### Example 11: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 20%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 120 g/L.

During the fermentation culture process, the RQ in the fermented broth and the specific growth rate were monitored in real time to perform the feedback control of the process in real time, the RQ and specific growth rate were controlled at the following levels by increasing or decreasing at least one of rotation speed, air flow and tank pressure, and when the RQ and specific growth rate could not be maintained within the predetermined ranges through the rotation speed, air flow and tank pressure, calcium hydroxide was added to the system, and the final concentration of calcium ions in the fermentation broth was controlled at 9 g/L:

1) during the fermentation period from hour 0 to hour 24, the RQ in the fermentation broth was controlled at 0.1˜1.1, and the fermentation process specific growth rate was controlled at 0.05˜0.8;

2) during the fermentation period from hour 24 to hour 48, the RQ in the fermentation broth was controlled at 0.2˜1.3, and the fermentation process specific growth rate was controlled at 0.2˜0.6;

3) during the fermentation period from hour 48 to hour 72, the RQ in the fermentation broth was controlled at 0.1˜0.8, and the fermentation process specific growth rate was controlled at 0.2˜0.5;

4) during the fermentation period from hour 72 to the end of fermentation, the RQ in the fermentation broth was controlled at 0.1˜0.4, and the fermentation process specific growth rate was controlled at 0.05˜0.3.

The fermentation was stopped when the growth rate of the product was significantly slowed down or the bacterial cells were lightly stained, and the glyceric acid content in the fermentation broth was determined by HPLC. When the fermentation was stopped, the glyceric acid content reached 155 g/L, and the conversion rate reached 56%.

### Example 12: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 12%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 120 g/L.

During the fermentation culture process, the redox potential in the fermented broth and the specific growth rate were monitored in real time to perform the feedback control of the process in real time, the redox potential and specific growth rate were controlled at the following levels by increasing or decreasing at least one of rotation speed, air flow and tank pressure, and when the redox potential and specific growth rate could not be maintained within the predetermined ranges through the rotation speed, air flow and tank pressure, calcium hydroxide was added to the system, and the final concentration of calcium ions in the fermentation broth was controlled at 9 g/L:

1) during the fermentation period from hour 0 to hour 24, the redox potential was controlled at −100˜50 mV, and the fermentation process specific growth rate was controlled at 0.05˜0.8;

2) during the fermentation period from hour 24 to hour 48, the redox potential was controlled at −300˜−50 mV, and the fermentation process specific growth rate was controlled at 0.2˜0.6;

3) during the fermentation period from hour 48 to hour 72, the redox potential was controlled at −250˜−100 mV, and the fermentation process specific growth rate was controlled at 0.2˜0.5;

4) during the fermentation period from hour 72 to the end of fermentation, the redox potential was controlled at −200˜−50 mV, and the fermentation process specific growth rate was controlled at 0.05˜0.3.

The fermentation was stopped when the growth rate of the product was significantly slowed down or the bacterial cells were lightly stained, and the glyceric acid content in the fermentation broth was determined by HPLC; when the fermentation was stopped, the glyceric acid content reached 155 g/L and the conversion rate reached 56%.

### Example 13: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 20%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 120 g/L.

During the fermentation culture process, the RQ and redox potential in the fermented broth as well as the fermentation process specific growth rate were monitored in real time to perform the feedback control of the process in real time, the RQ and redox potential as well as the fermentation process specific growth rate were controlled at the following levels by increasing or decreasing at least one of rotation speed, air flow and tank pressure, and when the RQ and redox potential as well as the fermentation process specific growth rate could not be maintained within the predetermined ranges through the rotation speed, air flow and tank pressure, calcium hydroxide was added to the system, and the final concentration of calcium ions in the fermentation broth was controlled at 9 g/L:

1) during the fermentation period from hour 0 to hour 24, the RQ in the fermentation broth was controlled at 0.1˜1.1, the redox potential was controlled at −100˜50 mV, and the fermentation process specific growth rate was controlled at 0.05˜0.8;

2) during the fermentation period from hour 24 to hour 48, the RQ in the fermentation broth was controlled at 0.2˜1.3, the redox potential was controlled at −300˜−50 mV, and the fermentation process specific growth rate was controlled at 0.2˜0.6;

3) during the fermentation period from hour 48 to hour 72, the RQ in the fermentation broth was controlled at 0.1˜0.8, the redox potential was controlled at −250˜100 mV, and the fermentation process specific growth rate was controlled at 0.2˜0.5;

4) during the fermentation period from hour 72 to the end of fermentation, the RQ in the fermentation broth was controlled at 0.1˜0.4, the redox potential was controlled at −200˜−50 mV, and the fermentation process specific growth rate was controlled at 0.05˜0.3.

The fermentation was stopped when the growth rate of the product was significantly slowed down or the bacterial cells were lightly stained, and the glyceric acid content in the fermentation broth was determined by HPLC; when the fermentation was stopped, the glyceric acid content reached 164 g/L, and the conversion rate reached 61%.

### Example 14: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 12%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 120 g/L.

During the fermentation culture process, the RQ and redox potential in the fermented broth as well as the fermentation process specific growth rate were monitored in real time to perform the feedback control of the process in real time, the RQ and redox potential as well as the fermentation process specific growth rate were controlled at the following levels by increasing or decreasing at least one of rotation speed, air flow and tank pressure, and when the RQ and redox potential as well as the fermentation process specific growth rate could not be maintained within the predetermined ranges through the rotation speed, air flow and tank pressure, calcium hydroxide was added to the system, and the final concentration of calcium ions in the fermentation broth was controlled at 9 g/L:

during the whole fermentation process, the RQ was controlled in the range of 0.1˜1.5, the redox potential was controlled in the range of −300˜50 mV, and the specific growth rate was controlled in the range of 0.05˜0.8.

The fermentation was stopped when the growth rate of the product was significantly slowed down or the bacterial cells were lightly stained, and the glyceric acid content in the fermentation broth was determined by HPLC; when the fermentation was stopped, the glyceric acid content reached 148 g/L, and the conversion rate reached 55%.

Compared with the staged control, the fermentation level and conversion rate of glycerol acid were lower.

### Example 15: Production Process for 160 m³ Tank Fermentation

S1. Seed activation: same as Example 1.

S2. Shake flask culture: same as Example 1.

S3. Seed tank culture: same as Example 1.

S4. Fermentation culture: A fermentation medium was prepared, adjusted to have a pH of 7.0, and then sterilized at 121° C. for 25 minutes, and the seed liquid of the seed tank was transferred to a 160 m³ fermentation tank at an inoculation amount of 20%, and the liquid filling volume was 90 m³. The initial culture conditions comprised: culture temperature of 30° C., rotation speed of 60 rpm, aeration ratio of 1.0 VVM, tank pressure of 0.05 MPa, the pH was controlled at about 6.5 by supplementing ammonia water during the culture process, and 550 g/L glucose and 4 g/L yeast extract powder were added in fed-batch manner during the culture process. Wherein, the components of the fermentation medium were as follows: potassium dihydrogen phosphate 8 g/L, sodium chloride 8 g/L, yeast extract powder 8 g/L, zinc chloride 0.15 g/L, manganese sulfate 0.15 g/L and glycerol 120 g/L.

During the fermentation culture process, the RQ and redox potential in the fermented broth as well as the fermentation process specific growth rate were monitored in real time to perform the feedback control of the process in real time, the RQ and redox potential as well as the fermentation process specific growth rate were controlled at the following levels by increasing or decreasing at least one of rotation speed, air flow and tank pressure, and when the RQ and redox potential as well as the fermentation process specific growth rate could not be maintained within the predetermined ranges through the rotation speed, air flow and tank pressure, calcium hydroxide was added to the system, and the final concentration of calcium ions in the fermentation broth was controlled at 9 g/L:

1) during the fermentation period from hour 0 to hour 12, the RQ in the fermentation broth was controlled at 0.1˜0.8, the redox potential in the fermentation broth was controlled at −50˜50 mV, and the specific growth rate was controlled at 0.05˜0.6;

2) during the fermentation period from hour 12 to hour 24, the RQ in the fermentation broth was controlled at 0.1˜1.1, the redox potential in the fermentation broth was controlled at −100˜−50 mV, and the specific growth rate was controlled at 0.2˜0.8;

3) during the fermentation period from hour 24 to hour 36, the RQ in the fermentation broth was controlled at 0.2˜1.3, the redox potential in the fermentation broth was controlled at −200˜−50 mV, and the specific growth rate was controlled at 0.2˜0.6;

4) during the fermentation period from hour 36 to hour 48, the RQ in the fermentation broth was controlled at 0.3˜1.0, the redox potential in the fermentation broth was controlled at −300˜−100 mV, and the specific growth rate was controlled at 0.3˜0.5;

5) during the fermentation period from hour 48 to hour 60, the RQ in the fermentation broth was controlled at 0.3˜0.8, the redox potential in the fermentation broth was controlled at −200˜−100 mV, and the specific growth rate was controlled at 0.2˜0.5;

6) during the fermentation period from hour 60 to hour 72, the RQ in the fermentation broth was controlled at 0.1˜0.6, the redox potential in the fermentation broth was controlled at −250˜−100 mV, and the specific growth rate was controlled at 0.15˜0.4;

7) during the fermentation period from hour 72 to hour 84, the RQ in the fermentation broth was controlled at 0.1˜0.4, the redox potential in the fermentation broth was controlled at −200˜−50 mV, and the specific growth rate was controlled at 0.1˜0.3;

8) during the fermentation period from hour 84 to the end of fermentation, the RQ in the fermentation broth was controlled at 0.1˜0.2, the redox potential in the fermentation broth was controlled at −150˜−50 mV, and the specific growth rate was controlled at 0.05˜0.15.

The fermentation was stopped when the growth rate of the product was significantly slowed down or the bacterial cells were lightly stained, and the glyceric acid content in the fermentation broth was determined by HPLC; when the fermentation was stopped, the glyceric acid content reached 170 g/L, and the conversion rate reached 65%.

Examples 16 to 45: Method for production of glyceric acid, which differed from Examples 1 to 15 in that the production strain was different, and the specific conditions and glyceric acid content in the fermentation broth and conversion rate were shown in Table 1.

TABLE 1

| | | | Glyceric acid | Conversion |
|---|---|---|---|---|
| Item | Production strain | Process control | content (g/L) | rate (%) |
| Example 16 | Gluconobacter | same as Example 1 | 122 | 49% |
| Example 17 | thailandicus | same as Example 2 | 142 | 54% |
| Example 18 | NBRC100600 | same as Example 3 | 130 | 49% |
| Example 19 | | same as Example 4 | 125 | 49% |
| Example 20 | | same as Example 5 | 149 | 54% |
| Example 21 | | same as Example 6 | 135 | 52% |
| Example 22 | | same as Example 7 | 123 | 48% |
| Example 23 | | same as Example 8 | 148 | 55% |
| Example 24 | | same as Example 9 | 132 | 51% |
| Example 25 | | same as Example 10 | 152 | 57% |
| Example 26 | | same as Example 11 | 156 | 58% |
| Example 27 | | same as Example 12 | 155 | 56% |
| Example 28 | | same as Example 13 | 163 | 62% |
| Example 29 | | same as Example 14 | 143 | 54% |
| Example 30 | | same as Example 15 | 169 | 65% |
| Example 31 | Gluconobacter | same as Example 1 | 125 | 48% |
| Example 32 | frateurii | same as Example 2 | 143 | 53% |
| Example 33 | NBR3262 | same as Example 3 | 134 | 51% |
| Example 34 | | same as Example 4 | 127 | 46% |
| Example 35 | | same as Example 5 | 149 | 55% |
| Example 36 | | same as Example 6 | 135 | 50% |
| Example 37 | | same as Example 7 | 125 | 51% |
| Example 38 | | same as Example 8 | 148 | 57% |
| Example 39 | | same as Example 9 | 136 | 52% |
| Example 40 | | same as Example 10 | 152 | 58% |
| Example 41 | | same as Example 11 | 154 | 59% |
| Example 42 | | same as Example 12 | 153 | 58% |
| Example 43 | | same as Example 13 | 163 | 61% |
| Example 44 | | same as Example 14 | 145 | 53% |
| Example 45 | | same as Example 15 | 171 | 64% |

Although the examples of the present invention have been shown and described above, it should be understood that the above examples are exemplary and should not be construed as limiting the present invention, and those of ordinary skill in the art will make variations, modifications, substitutions, and alterations to the above-described examples without departing from the principles and spirit of the present invention.

What is claimed is:

1. A method for controlled production of glyceric acid, the method comprising: during a glyceric acid fermentation process, performing real-time and online monitoring of at least one of a respiratory quotient and redox potential in a fermented broth as well as a fermentation process specific growth rate, and controlling values thereof within the following ranges: 0.1˜1.5 for respiratory quotient, −300˜50 mV for redox potential, and 0.05˜0.8 for specific growth rate.

2. The method for controlled production of glyceric acid according to claim 1, wherein:
the respiratory quotient is controlled in 4 stages, and the control method is as follows: during the fermentation period from hour 0 to hour 24, the respiratory quotient in the fermentation broth is controlled at 0.1˜1.1; during the fermentation period from hour 24 to hour 48, the respiratory quotient in the fermentation broth is controlled at 0.2˜1.3; during the fermentation period from hour 48 to hour 72, the respiratory quotient in the fermentation broth is controlled at 0.1˜0.8; and during the fermentation period from hour 72 to the end of fermentation, the respiratory quotient in the fermentation broth is controlled at 0.1˜0.4.

3. The method for controlled production of glyceric acid according to claim 1, wherein:
the redox potential is controlled in 4 stages, and the control method is as follows: during the fermentation period from hour 0 to hour 24, the redox potential in the fermentation broth is controlled at −100˜50 mV; during the fermentation period from hour 24 to hour 48, the redox potential in the fermentation broth is controlled at −300˜−50 mV; during the fermentation period from hour 48 to hour 72, the redox potential in the fermentation broth is controlled at −250˜−100 mV; and during the fermentation period from hour 72 to the end of fermentation, the redox potential in the fermentation broth is controlled at −200˜−50 mV.

4. The method for controlled production of glyceric acid according to claim 1, wherein:
the specific growth rate is controlled in 4 stages, and the control method is as follows: during the fermentation period from hour 0 to hour 24, the specific growth rate is controlled at 0.05˜0.8; during the fermentation period from hour 24 to hour 48, the specific growth rate is controlled at 0.2˜0.6; during the fermentation period from hour 48 to hour 72, the specific growth rate is controlled at 0.2˜0.5; and during the fermentation period from hour 72 to the end of fermentation, the specific growth rate is controlled at 0.05˜0.3.

5. The method for controlled production of glyceric acid according to claim 1, wherein the numerical ranges of the respiratory quotient and redox potential in the fermented broth as well as the fermentation process specific growth rate are controlled by adjusting at least one of rotation speed, air flow, and tank pressure, and when the numerical ranges of the respiratory quotient, the redox potential, and the fermentation process specific growth rate cannot be maintained within predetermined ranges by adjusting the rotation speed, the air flow and the tank pressure, a feed solution is supplemented, and the feed solution is at least one selected from the group consisting of calcium oxide, calcium hydroxide and calcium carbonate.

6. The method for controlled production of glyceric acid according to claim 1, wherein the strain used for the glyceric acid fermentation is at least one selected from the group consisting of Acetobacter tropicalis, Gluconobacter thailandicus and Gluconobacter frateurii.

7. The method for controlled production of glyceric acid according to claim 1, wherein the production process of glyceric acid comprises the following steps:
S1. Seed activation: A plate medium is prepared, its pH value is adjusted to 5.5 to 7.5, and then it is sterilized at 121˜123° C. for 20˜30 minutes; the components of the plate medium are as follows: sodium chloride 1˜10 g/L, peptone 1˜15 g/L, yeast extract powder 1˜15 g/L and agar powder 10˜20 g/L; a bacterial liquid is pipetted from a seed preservation tube and subjected to gradient dilution, the diluted bacterial suspension is pipetted and transferred to the sterilized plate medium, and cultured at 28˜38° C. for 2˜5 days to obtain matured single colonies;
S2. Shake flask culture: A shake flask medium is prepared, its pH value is adjusted to 5.5 to 7.5, and then it is sterilized at 121˜123° C. for 20˜30 minutes; the components of the shake flask medium are as follows: sodium chloride 1˜10 g/L, peptone 1˜15 g/L and yeast extract powder 1˜15 g/L; 1˜20 single colonies are picked from the matured plate medium and inoculated to the sterilized shake flask medium, the shake flask culture conditions comprise: culture temperature of 28˜38° C., rotation speed of 150˜250 rpm, and culture cycle of 4˜48 hours; when the wet weight of the bacteria reaches 1˜20 g/L, it is transferred to a seed tank, and the inoculation amount is controlled at 0.1˜10%;
S3. Seed tank culture: A seed medium is prepared, its pH value is adjusted to 5.5 to 7.5, and then it is sterilized at 121˜123° C. for 20 to 30 minutes; the components of the seed medium are as follows: glucose 1˜15 g/L, sodium chloride 1˜10 g/L, peptone 1˜15 g/L and yeast extract powder 1˜15 g/L; the seed tank culture conditions comprise: culture temperature of 28˜38° C., tank pressure of 0.025˜0.08 MPa, aeration ratio of 0.2˜2 VVM, rotation speed of 100˜500 rpm, culture cycle of 4˜48 hours, when the wet weight of the bacteria reaches 1˜20 g/L, it is transferred to a fermentation tank, and the inoculation amount is controlled at 5˜30%; and
S4. Fermentation culture: A fermentation medium is prepared, its pH value is adjusted to 5.5˜7.5, and then it is sterilized at 121˜123° C. for 20˜30 minutes; the components of the fermentation medium are as follows: potassium dihydrogen phosphate 1˜10 g/L, sodium chloride 1˜10 g/L, yeast extract powder 1˜15 g/L, zinc chloride 0.005˜0.5 g/L, manganese sulfate 0.0001˜0.5 g/L and glycerol 20˜300 g/L; the fermentation culture conditions comprise: culture temperature of 28˜38° C., tank pressure of 0.02˜0.08 MPa, aeration ratio of 0.2˜2 VVM, and rotation speed of 50˜1000 rpm; during the fermentation culture process, an alkali is added to control the pH value of the fermentation system at 3.5˜7.5, a fed-batch medium is supplemented to control the carbon source concentration at 0.1˜100 g/L and the nitrogen source concentration at 0.01˜2 g/L in the fermentation system.

8. The method for controlled production of glyceric acid according to claim 7, wherein, in step S4, the alkali is sodium hydroxide and/or ammonia water; the carbon source is at least one selected from the group consisting of glucose, sucrose and glycerol; and the nitrogen source is at least one selected from the group consisting of ammonium sulfate, yeast extract and peptone.

9. The method for controlled production of glyceric acid according to claim 7, wherein, in step S4, the components of the fed-batch medium are as follows: glycerol concentration 200˜600 g/L, ammonia water 200˜280 g/L, sodium hydroxide 100˜400 g/L, ammonium sulfate 1˜50 g/L, and yeast extract powder 1˜50 g/L.

10. The method for controlled production of glyceric acid according to claim 7, wherein, in step S4, when the product growth rate is slowed down or the staining of bacterial cells is light, which is used as a standard for stopping the fermentation, HPLC is used to measure the glyceric acid concentration in the fermentation broth.

11. The method for controlled production of glyceric acid according to claim 1, wherein:
the respiratory quotient is controlled in 8 stages, and the control method is as follows: during the fermentation period from hour 0 to hour 12, the respiratory quotient in the fermentation broth is controlled at 0.1˜0.8; during the fermentation period from hour 12 to hour 24, the respiratory quotient in the fermentation broth is controlled at 0.1˜1.1; during the fermentation period from hour 24 to hour 36, the respiratory quotient in the fermentation broth is controlled at 0.2˜1.3; during the fermentation period from hour 36 to hour 48, the respiratory quotient in the fermentation broth is controlled at 0.3˜1.0; during the fermentation period from hour 48 to hour 60, the respiratory quotient in the fermentation broth is controlled at 0.3˜0.8; during the fermentation period from hour 60 to 72, the respiratory quotient in the fermentation broth is controlled at 0.1˜0.6; during the fermentation period from hour 72 to hour 84, the respiratory quotient in the fermentation broth is controlled at 0.1˜0.4; during the fermentation period from hour 84 to the end of fermentation, the respiratory quotient in the fermentation broth is controlled at 0.1˜0.2.

12. The method for controlled production of glyceric acid according to claim 1, wherein:
the redox potential is controlled in 8 stages, and the control method is as follows: during the fermentation period from hour 0 to hour 12, the redox potential in the fermentation broth is controlled at −50˜50 mV; during the fermentation period from hour 12 to hour 24, the redox potential in the fermentation broth is controlled at −100˜−50 mV; during the fermentation period from hour 24 to hour 36, the redox potential in the fermentation broth is controlled at −200˜−50 mV; during the fermentation period from hour 36 to hour 48, the redox potential in the fermentation broth is controlled at −300˜−100 mV; during the fermentation period from hour 48 to hour 60, the redox potential in the fermentation broth is controlled at −200˜−100 mV; during the fermentation period from hour 60 to hour 72, the redox potential in the fermentation broth is controlled at −250˜−100 mV; during the fermentation period from hour 72 to hour 84, the redox potential in the fermentation broth is controlled at −200˜−50 mV; during the fermentation period from hour 84 to the end of fermentation, the redox potential in the fermentation broth is controlled at −150˜−50 mV.

13. The method for controlled production of glyceric acid according to claim 1, wherein:
the specific growth rate is controlled in 8 stages, and the control method is as follows: during the fermentation period from hour 0 to hour 12, the specific growth rate is controlled at 0.05˜0.6; during the fermentation period from hour 12 to hour 24, the specific growth rate is controlled at 0.2˜0.8; during the fermentation period from hour 24 to hour 36, the specific growth rate is controlled at 0.2˜0.6; during the fermentation period from hour 36 to hour 48, the specific growth rate is controlled at 0.3˜0.5; during the fermentation period from hour 48 to hour 60, the specific growth rate is controlled at 0.2˜0.5; during the fermentation period from hour 60 to hour 72, the specific growth rate is controlled at 0.15˜0.4; during the fermentation period from hour 72 to hour 84, the specific growth rate is controlled at 0.1˜0.3; during the fermentation period from hour 84 to the end of fermentation, the specific growth rate is controlled at 0.05˜0.15.

14. The method for controlled production of glyceric acid according to claim 5, wherein the feed solution is used in an amount such that the final concentration of calcium ions in the fermentation broth is 0.5 to 15 g/L.

15. The method for controlled production of glyceric acid according to claim 2, wherein:
the redox potential is controlled in 4 stages, and the control method is as follows: during the fermentation period from hour 0 to hour 24, the redox potential in the fermentation broth is controlled at −100˜50 mV; during the fermentation period from hour 24 to hour 48, the redox potential in the fermentation broth is controlled at −300˜−50 mV; during the fermentation period from hour 48 to hour 72, the redox potential in the fermentation broth is controlled at −250˜−100 mV; and during the fermentation period from hour 72 to the end of fermentation, the redox potential in the fermentation broth is controlled at −200˜−50 mV.

16. The method for controlled production of glyceric acid according to claim 15, wherein:
the specific growth rate is controlled in 4 stages, and the control method is as follows: during the fermentation period from hour 0 to hour 24, the specific growth rate is controlled at 0.05˜0.8; during the fermentation period from hour 24 to hour 48, the specific growth rate is controlled at 0.2˜0.6; during the fermentation period from hour 48 to hour 72, the specific growth rate is controlled at 0.2˜0.5; and during the fermentation period from hour 72 to the end of fermentation, the specific growth rate is controlled at 0.05˜0.3.
